(19) United States
(12) Patent Application Publication
(10) Pub. No.: US 2025/0195422 A1
(43) Pub. Date: Jun. 19, 2025

(54) USER-ADAPTIVE ORAL POUCH AND METHOD OF USE

(71) Applicant: Pocket Tea, LLC, Weston, CT (US)

(72) Inventor: Daniel Victor, Weston, CT (US)

(21) Appl. No.: 19/064,022

(22) Filed: Feb. 26, 2025

Related U.S. Application Data
(63) Continuation of application No. PCT/US2023/072611, filed on Aug. 23, 2023.
(60) Provisional application No. 63/402,276, filed on Aug. 30, 2022.

(51) Int. Cl. A61K 9/00; A24B 15/28

(52) U.S. Cl. CPC A61K 9/009; A24B 15/285

(57) ABSTRACT

A consumer-customizable oral pouch product of liquid-permeable material is sold containing a carrier material capable of absorbing or adsorbing active materials but containing no active material. Consumers adapt the pouch to their individual tastes by adding active materials of their choice such as medicaments, pharmaceuticals and stimulants.

### CROSS REFERENCE TO RELATED APPLICATIONS

This is a Continuation of International Application No. PCT/US2023/072611 filed, Aug. 22, 2023, which claims benefit to U.S. Provisional Application No. 63/402,276, titled “Oral Pouch and Method of Use”, filed Aug. 30, 2022, the disclosure in which is hereby incorporated herein by reference in its entirety.

### TECHNICAL FIELD

This disclosure pertains generally to oral pouches and, more particularly, to improvements in the marketing and use of pouches designed to release medicaments, stimulants, and other active ingredients when the pouch and its contents interact with saliva in the mouth of a user.

### BACKGROUND

Conventional oral pouch products comprise an active ingredient, typically in granular or other small particle form, encased in a sealed pouch of porous, material. The active ingredients in such oral pouch products, such as a flavorants, nicotine, a pharmaceutical, etc., are dispersed in a carrier that absorbs or adsorbs the active ingredient and releases or otherwise delivers it for its final use, e.g., when moistened by saliva in the mouth of the consumer or end user. A typical carrier material for this purpose is microcrystalline cellulose (MCC), used alone or in combination with other material, which has little or no taste or smell that can be sensed or otherwise perceived by a typical consumer. Conventional oral pouch products are designed and configured by the manufacturer to accomplish a predetermined common result or effect for their end users, namely facilitating the mixing of saliva with the contents of the pouch so that specific elements of the active ingredients borne by the carrier inside the pouch exit the pouch through pores, holes, interstices or similar openings in the pouch material with a precise, specific and intended effect on the user. That effect is entirely and exclusively designed and intended by the manufacturer of the pouch product; that is, the pouch is intended to be taken from its package and placed in the user's mouth, and that is substantially the extent of the user's control of the effects of the product. Otherwise stated, the pouch product manufacturer creates a finished product designed for interaction with the end user's taste buds, olfactory senses, mucosal lining, stomach, small intestine and/or other potential areas for absorption or adoption by the user's body. The manufacturer-designed finished product is not formulated, intended, or designed to be user-adaptive (i.e., customized by the end user); that is, the product is intended to be used with the precise dose and specific type of active ingredients, the amount and type of flavor, and even the moisture level predetermined exclusively by the manufacturer. Once sealed by the manufacturer, the typical pouch is a finished product having the manufacturer-determined effect when used by a consumer.

Even in cases where pouch product manufacturers have sought to develop, or have developed, oral pouch products with one or more coatings on the exterior of the pouch, or where the manufacturer has added one or more ingredients to the exterior of the oral pouch after it has been sealed, these coatings and ingredients are added by the manufacturer, in its sole determination, to the sealed pouch as a part of the manufacturing process. Thus, these oral pouch products are also designed and intended for the end user to use the product strictly as manufactured and predetermined by the manufacturer.

Alternatively, it has been, and continues to be, possible for end users to purchase an empty tea bag-like pouch and fill it with herbs, leaves and/or other ingredients. In these instances, the manufacturer is providing nothing but a physically empty “vessel” to be filled with anything for the purpose of making “at home” products as diverse as tea to potpourri, not even exclusively for ingestion. Such made at home products invariably require limited, or no, expertise.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In one aspect, the present disclosure pertains to a method comprising marketing an oral pouch product of liquid-permeable material containing a carrier for active material but no active material capable of being absorbed by or adsorbed on the carrier material and distributing the oral pouch product to consumers to add active material of their choice to the oral pouch product.

In another aspect the present disclosure is directed to a method of delivering active materials to the mouth of a consumer by means of a consumer-customizable pouch, the method comprising: offering for sale an oral pouch of saliva-permeable pouch material containing a carrier capable of sorbing different active materials, wherein the pouch as sold contains none of the active materials; and by the consumer: adding an active material of the consumer's choice to the pouch such that the added active material is sorbed by the carrier in the pouch; and placing the pouch with the sorbed active material in the consumer's mouth.

In another aspect, the present disclosure is directed to an oral pouch product of liquid-permeable pouch material for delivering active material to the mouths of consumers when contacted by saliva, the oral pouch product as sold to consumers containing a carrier capable of absorbing or adsorbing active material but no active material, wherein consumers add active material of their choice after purchase.

In the described method and product, instructions may be provided with the product, or via a web site, or via word mouth, or via a specific product name that is suggestive of instructions, such instructions directing or suggesting to consumers to add active material and/or flavorant as liquid applied to and through the pouch material, and may further direct consumers to add that material as drops of liquid to the pouch material.

The products and methods described herein differ from prior oral pouch products and methods, both in manufacture and use, in that the product is a sealed pouch containing a carrier for active ingredients but having no active ingredients. In addition, the carrier may be provided without taste or aroma capable of being sensed by consumers except incidental, minimally distinguishable tastes and odors inherent in some carrier ingredients. The carrier comprises one or more ingredients formulated and designed specifically to absorb or adsorb active ingredients such as medicaments, nicotine, cannabinoids, THC, caffeine, essential oils, or other active ingredients, but those active ingredients are not included in or on the pouch product as sold. Instead, active ingredients of the consumer's choice are added by the consumer when using the pouch. The carrier may be entirely conventional and of any type used in prior oral pouch products where the active ingredient or ingredients are sorbed on or in the carrier during manufacture.

It is important to stress that, for the oral pouch product described herein, the active product is selected by the end users who add the active product(s) of their choice to the carrier. Instructions may be provided with the oral pouch products, or otherwise, directing consumers to add the active ingredients. This may be done by applying the active ingredients in liquid form to the exterior of the pouch so as to be absorbed or adsorbed partially by the pouch material but primarily by the carrier ingredient(s) contained inside the pouch after passing through the pouch material. In this way the pouch is adaptive to the tastes/needs of, and is in practical effect fully customizable by, the end user. In other words, the pouch and its carrier contents serve to: (a) absorb or adsorb the end user's desired personalized or customized active ingredients, such as a medicament, nicotine, caffeine, essential oils, flavorants, etc., or other active additive ingredients when applied by the user; and then (b) deliver the received additive ingredients to the user's mouth when exposed to saliva.

### BRIEF DESCRIPTION OF THE DRAWINGS

By way of example, specific illustrative embodiments of the present disclosure will now be described with reference to the accompanying drawings.

FIG. 1 is a top plan view of a typical oral pouch that may be used in accordance with the principles described herein.

FIG. 2 is a side view in perspective of the oral pouch of FIG. 1.

FIG. 3 is a bottom view in plan of the pouch of FIG. 1.

FIG. 4 is a side view in perspective illustrating application of active ingredients to the pouch of FIG. 1.

### DETAILED DESCRIPTION

The products and methods embodying the principles of the present disclosure are described more fully hereinafter with reference to the accompanying drawings, in which several exemplary embodiments are shown. It will be readily understood that the components of the embodiments as generally described herein and illustrated in the appended drawings may be arranged and designed in a wide variety of different configurations. Thus, the following more detailed description of various embodiments, as represented in the drawings, is not intended to limit the scope of the present disclosure but is merely representative of various embodiments. While the various aspects of the embodiments are presented in the drawings, the drawings are not necessarily drawn to scale unless specifically indicated.

The principles described herein may be embodied in other specific forms without departing from the spirit or essential characteristics of those principles. The described embodiments are to be considered in all respects only as illustrative and not restrictive. The scope of the inventions is, therefore, indicated by the appended claims rather than by this detailed description. All changes which come within the meaning and range of equivalency of the claims are to be embraced within their scope.

Reference throughout this specification to features, advantages, or similar language does not imply that all the features and advantages that may be realized with the disclosed embodiments should be or are in any single embodiment. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosed principles may be combined in any suitable manner in one or more embodiments. One skilled in the relevant art will recognize, in light of the description herein, that the principles can be practiced without one or more of the specific features or advantages of a particular embodiment. In other instances, additional features and advantages may be recognized in certain embodiments that may not be present in all the embodiments.

Reference throughout this specification to “one embodiment”, “an embodiment”, or similar language means that a particular feature, structure, or characteristic described in connection with the indicated embodiment is included in at least one embodiment. Thus, the phrases “in one embodiment”, “in an embodiment”, and similar language throughout this specification may, but do not necessarily, all refer to the same embodiment.

As used herein the following terms shall be considered to have the meanings described:
(a) A “carrier” means a material such as, for example, microcrystalline cellulose (MCC), or other naturally occurring or synthetic materials that are commonly used in oral pouches, which are capable of absorbing or adsorbing active ingredients to be added by consumers and then releasing them in the presence of saliva. Optionally, a carrier may also include:
   (i) a stabilization and emulsification component, such as modified food starch;
   (ii) a prebiotic, such as an inulin;
   (iii) a sweetening enhancer with no calories, such as sucralose; and/or
   (iv) a humectant (e.g., glycerin; MCT coconut oil).
      A “carrier” may in some cases be an excipient that surrounds applied active ingredients, carries and protects them, and releases or otherwise delivers them for final use when exposed to saliva in the mouth of an end user of the pouch product that is the subject of this disclosure.
(b) An “active ingredient” or “active material” means flavorants, medicaments, pharmaceuticals, stimulants such as nicotine, caffeine, essential oils, cannabinoids, THC and the like, that when exposed to saliva in the mouth of an end user deliver an intended effect through the mucosal lining of the oral cavity, such as a taste, a smell, a remedial effect, a stimulative effect, a medicinal effect, a psychopharmacological effect, a flavor, or the like. In this regard, “flavorant” as used herein means an active ingredient to be added by the consumer to impart flavor, whereas “flavoring” as used herein means a taste of a constituent of the carrier that is incidental to the primary function of that constituent in the carrier.

In one aspect, the present disclosure pertains to a sealed oral pouch product containing an adaptive carrier that is receptive to and designed to absorb or adsorb active ingredients added prior to use by the end user, thus allowing for personalization and customization by the end user without requiring the end user to have an expertise that is typically reserved for use during manufacture by professionals with commercial blending equipment.

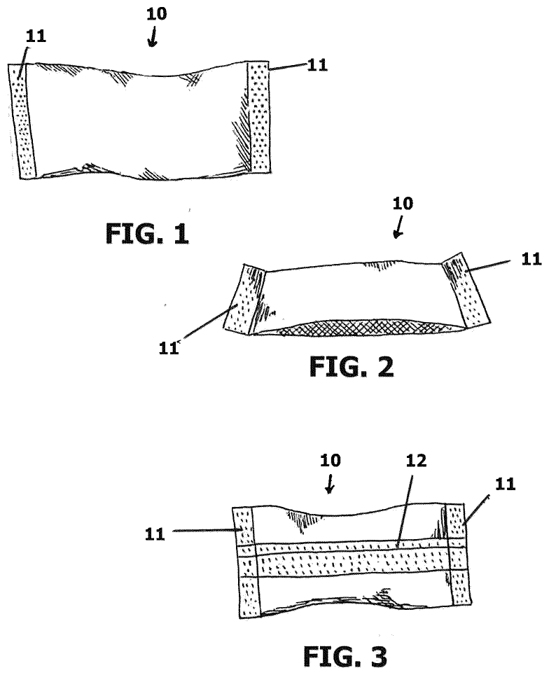

An oral pouch 10 incorporating the principles of this disclosure is illustrated in FIGS. 1, 2 and 3 and, as sold to a consumer, contains a suitable carrier of the type described. The pouch itself is conventional and may be made of a nonwoven or other saliva-porous material that contains the granulated or other small particle materials described herein and is permeable to water, saliva and oils such as propylene glycol, vegetable glycerin, MCT oil-based fluids, etc. The composition/construction of the water-permeable [same issue as directly above] fabric pouch 10 may be varied as noted herein. For example, the pouch material may be of a type considered to be similar in character to the mesh-like type of material that is used for tea bags or for smokeless tobacco products. Typical pouch materials of this kind are disclosed in U.S. Pat. No. 9,126,704(Williams), U.S. Pat. No. 11,019,840 (Sebastian et al.), and U.S. Pat. No. 11,344,057 (Chapman et al,), the entire disclosures in which are incorporated herein by reference. Functionally, as noted, the pouch material must retain the pre-customized carrier within the pouch and permit passage of liquid into and out of the pouch. The example pouch 10 of FIGS. 1-3 has transverse seals 11 at opposite ends and a longitudinal seal 12 extending therebetween, but other pouch configurations may be used to the extent they provided the features and functions described herein.

Pouch 10 is sized and configured to fit comfortably in a consumer's mouth, preferably between the cheek or lip and gum, so that a consumer may passively enjoy or orally manipulate the pouch. Preferably, but not as a limitation, the pouch can be 6 mm to about 30 mm in width, approximately 12 mm to about 50 mm in length and, with contents therein as described below, approximately 1.5 mm to about 20 mm thick. The pouch shape is preferably rectangular, quadrilateral, cylindrical, or oblong; other shapes may be utilized so long as the pouch fits comfortably and discreetly in a consumer's mouth.

The pouch product 10, as manufactured and sold or distributed to consumers, contains a measured or predetermined amount of solid carrier material, typically in granulated or powdered form, suitable for sorbing active ingredients to be added to the pouch by the consumer or end user after sale or distribution of the product. The carrier material may be, for example, microcrystalline cellulose, cornstarch, beet pulp fiber, silicon dioxide, calcium polycarbophil, or calcium carbonate or combinations thereof. Importantly, for the concepts and principles described herein, the pouch as sold or distributed contains no active ingredients. Stated otherwise the contents of the pouch as sold, when mixed with saliva in an end user's mouth, has no significant physical or biochemical effect on the end user.

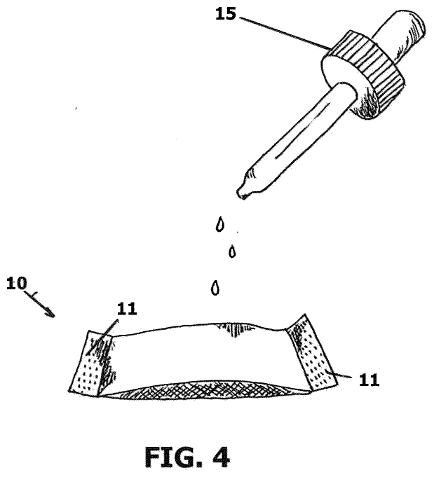

As sold, the pouch is packaged in a container containing multiple similar or identical pouches and may be accompanied by instructions for the end user to add an active ingredient, e.g., a medicament, flavorant, pharmaceutical or stimulant of the end user's choice. Such instructions may appear on a package containing one or more pouches or on an instruction sheet included in such package. Alternatively, such instructions may appear on a web site or in social media or may be passed along by word of mouth by other consumers or may be in audio/visual form. In any case, the instructions inform the end user that the product is customizable and how to add the active ingredient, for example, by adding it in liquid form onto and through the pouch material from a dropper 15 as shown in FIG. 4. Alternatively, the liquid active ingredient(s) may be added by brushing, spraying or pouring it onto the pouch material, by dipping the pouch into the liquid, etc. Some of the active ingredients thusly added may be sorbed by the pouch material but are primarily absorbed by or adsorbed on the carrier that was included in the pouch by the pouch product manufacturer.

Once the end user adds the active ingredients and is ready to use the resulting product, the pouch is placed in the end-user's mouth where contact with saliva cause the active ingredient(s) to be released from the carrier material and transported through the liquid-permeable fabric pouch into the end user's mouth. The end user may thusly benefit from the saliva-laden active ingredients by swallowing it or otherwise absorbing it through oral mucosa. The pouch itself is not designed to be swallowed. It may be moved around the user's mouth for manipulative purposes but not chewed so as to substantially tear or otherwise perforate the pouch and allow the internal contents to spill into the user's mouth. After a time suitable for use/enjoyment by the user (e.g., anywhere from about three minutes to about an hour), substantial amounts of the released active ingredients may be ingested and/or absorbed by the user and the pouch may be removed from the user's mouth.

As described, the end user applies the active ingredients as a liquid directly to and through the pouch material; that is, the end user does not need to, and should not, open the pouch to insert ingredients because the ability to re-seal the pouch along its length and ends typically requires high pressure and heat. The carrier already sealed within the pouch is created and designed for total customization by the end user, and the nature of the pouch with its absorptive properties and interstices that allow for the absorption of ingredients by the formula within, allows for full customization without opening by the end user. In addition, and given that a high-end pouch machine can create 1000 pouches per minute or more with pouches manufactured in a string and cut as much as 1000 times per minute or more by the cutting blade, the pouch product is the result of a high degree of expertise, expense and technological know-how that is not readily possessed by or available to end users.

To be clear, the pouch product upon leaving the manufacturer's facility is not intended to be used “as is” and it would serve no purpose if it were. The pouch product leaves the manufacturer fully-commercially packaged and is intended to be taken out of the commercial package by the end user. In this way the present pouch product is the exact opposite of the ready-to-use typical pouch product which is intended for no user-adaptation whatsoever. Neither is the present pouch product an “empty tea bag” where the end user is responsible for supplying 100% of the ingredients. The present pouch product does not dictate to the end user flavor(s), dosage of active ingredients or even which single or multiple active ingredient(s) to use; nor does it specify moisture levels. The present product comprises an oral pouch that is intentionally formulated, designed and manufactured so as to enable an end user to customize the product to that user's taste and/or needs.

Unlike typical oral pouch products, the present pouch product also does not dictate to end users that all the pouches within a retail package must be exactly the same or used/enjoyed in the same way. Typically, all of the prior pouches contained in a retail package contain the same active ingredient(s), dosage, moisture level and flavor. The present pouch product, containing no active ingredients, allows the end user to customize each individual pouch within a retail package with no predetermination from manufacturer. For some end users, a desire for a specific attribute of a typical prior art pouch product (e.g., flavor) can be frustrated by having to accept other attributes that are less desirable. That is, a typical prior oral pouch product may have the desired flavor, for example, but may be too dry for that customer to use the product comfortably.

The principles disclosed herein do not dictate to end users that they must purchase specific flavorants in specific amounts of an active ingredient; whether that active ingredient be caffeine, a cannabinoid, nicotine, an essential oil or another ingestible and/or absorbable ingredient. Moreover, the present disclosure does not dictate that each packaging unit sold must have only a specific flavor and specific active ingredient in a specific amount or a specific moisture level.

Rather, consumers are allowed to customize a single pouch, with qualities such as, but not limited to, the flavor, moisture level and type and dosage of an active ingredient. As importantly, the principles disclosed herein do not require the end user to include any particular active ingredient.

The ability to uniquely customize even a single pouch assists end users who want to gradually reduce use of a specific active ingredient such as nicotine or caffeine but are on a limited budget. In prior pouch products containing a nicotine or caffeine dose set by the manufacturer, for example, a user purchases a container of twenty oral nicotine pouches, all of which contain the same flavor and nicotine dose. With the pouch product disclosed herein, an end user can customize some pouches with a greater nicotine or caffeine strength and others with a lower dose, thus permitting gradual reduction of the dose and dependency with successive pouches used. With prior art products a customer may purchase different containers of different active ingredient strengths and flavors as an alternative to the customization disclosed herein, but this may require a customer to purchase five or ten products instead of one. In addition to the exorbitant cost associated with purchasing those different products, which in and of itself may be enough to discourage a person of modest means from trying to gradually reduce their nicotine use, carrying around five or ten different products is totally impractical. An end user of the disclosed pouch product may even add only flavorant to the pouch so as to receive the satisfaction and feel of a pouch without any other addictive active ingredients. The customization feature also enables consumers to use and enjoy an oral pouch with flavors that are not currently available in pouch form.

The disclosed pouch product also permits an end user to customize the pouch as a weight loss, appetite alternative or appetite suppression product where only a flavorant is added, or a flavorant and other nutraceutical ingredients in liquid form can be added, thus satisfying a flavor and sensory craving (and receiving the benefit of nutraceutical ingredients, if any) but providing zero or minimal calories. For an end user looking to use the pouch product disclosed herein as a tool to satisfy sensory cravings and who seeks to eat less, the ability to flavor individual pouches with a liquid that comes in flavors from hot chocolate to salted caramel is extremely attractive. In addition, the customization capability allows each pouch within a package to be flavored differently to satisfy different cravings during the day or evening.

The presently disclosed pouch also provides a unique opportunity for interaction with other products, as the pouch can be flavored for a specific need or restriction. For example, an end user, who desires the flavor of an “Arnold Palmer” beverage (i.e., a combination of lemonade and iced tea favored by the famous golfer) but does not want all the sugar in the lemonade, may flavor a single pouch with lemonade flavorant and place the pouch in his/her mouth prior to drinking the iced tea. The iced tea would interact with the lemonade flavored pouch providing the taste of an Arnold Palmer beverage without the sugar. This could also be an important attribute of the pouch product for people with dietary restrictions who want to enjoy a drink without, for example, ingesting sugar.

The unique capability offered by the present pouch product for the end user to customize the pouch with a variety of flavors, singular or in combination, is enhanced by the fact that there is virtually no limit to the products with which it can interact. The pouch is not designed to be used only with, by way of example, e-liquids from a specific company; the pouch is designed to be adaptably agnostic. What can be added can be any combination of flavorants or other active ingredients to offer a unique customization by the end user. A basic feature of the present pouch is that it is intended and designed specifically for customization by the end user.

In one embodiment the present pouch offers a healthier alternative to people who use electrical devices that vaporize nicotine because they can add the same e-liquids or vape juice (i.e., the same flavors and nicotine dose) to the pouch and get a similar or identical result but in a non-respiratory way. The e-liquids or vape juice are comprised typically of some combination of propylene glycol, vegetable glycerin, nicotine and flavor (although many e-liquids also offer options with zero nicotine), and there is evidence to suggest that adding them to the pouch is a safer method of ingestion than inhalation. The present pouch product is specifically formulated to allow the consumer/end user to create an alternative to vaping with similar flavors and nicotine strengths. The present pouch also allows people who vape to use the same flavors and doses they prefer in environments where vaping is prohibited, such as airplanes, trains, educational and social institutions, restaurants, sports venues, etc.

The method of use of the present pouch is also different from that of conventional oral pouch products which are intended to be taken from the commercial packaging and put directly into the mouth of the end user. Because the pouch is provided to the end user without active ingredients, the end user is required to customize the product before use. Unlike other pouch products, the present pouch is never to be taken directly from the commercial packaging and placed in the mouth of the end user. Anyone who wants a product that can be used as purchased, and used only as purchased, has a myriad of product options. While it is possible with such prior pouch products for someone to add an additional flavorant or other active ingredient to the active ingredient provided by the pouch manufacturer, it is not possible to fully customize such pouch without the ingredients and their amounts dictated by the manufacturer.

In another embodiment, the present pouch product may allow end users to add active ingredients such as, but not limited to, tinctures that are typically taken sublingually but which can be released more slowly through the present pouch product. Instead of a dose under the tongue, for example, the present pouch allows for a more time-released method of ingestion or absorption via the mucosal lining or sublingually. Further, absorption sublingually or through the mucus membranes is faster and more efficient than ingestion via the gastrointestinal tract.

Ingredients provided in the present pouch product as manufactured and sold are in dry granulated or powdered form configured to be retained in the pouch as sold. Those ingredients are designed to serve as a carrier or excipient for the active ingredients added by the end user, and may include, individually or in combination, any of the following (percentages stated by weight): microcrystalline cellulose in a range from 0% to 100%, modified food starch in a range from 0% to 50%, soluble prebiotic dietary fiber in a range from 0% to 40%,, sodium bicarbonate in a range from 0% to 20%, propylene glycol in a range from 0% to 40%, vegetable glycerin in a range from 0% to 30%, a gum base in particle form in a range from 0% to 50%, gelatin in powder form in a range from 0% to 100%, bone meal in a range from 0% to 100%, other cellulose material in a range from 0% to 100% and the optional addition of a very small quantity of sweetener or sweetening enhancers such as sucralose in encapsulated or non-encapsulated form in a range from 0% to 38%, xylitol in a range from 0% to 36%, Erythritol in a range from 0% to 34%, coconut oil in a range from 0% to 41%.

If a flavorant is the ingredient to be added by the end user, it may be added to the exterior of the pouch (on one or both sides) and absorbed into the pouch formula, or in the same or alternate form, injected through the weave or interstices or gaps in the nonwoven or similarly porous material, in a liquid form or included in a liquid carrier. Individual or combined active ingredients and flavorants may be added into the same oral pouch.

In another embodiment the end user may add a liquid for absorption to the exterior of the pouch which could be comprised of (by weight): propylene glycol between 0% and 100%; vegetable glycerin between 0% and 100%; flavorant between 0% and 40%; and nicotine 0% and 25%.

In one or more functional embodiments the pouch may be a nonwoven material made from viscose fibers with an acrylic binder which binds the material fibers and seals the pouch. The ingredients inside the pouch are comprised of (by weight): microcrystalline cellulose in granular form (84.6%), modified food starch (4%), soluble prebiotic dietary fiber (2%), sucralose (1.4%), xylitol (4%), and erythritol (4%). The microcrystalline cellulose in this embodiment is non-water soluble and holds and releases flavor and active ingredients but does not dissolve in the mouth because it does not dissolve with saliva, thereby allowing the pouch form to hold its shape. In this embodiment, and in some other but not all embodiments, sweeteners may be included, as in the chewing gum industry, to extend the customized flavorant added by the end user.

In use, if for example an end user desires to use the pouch with nicotine, the end user may add a desired amount of nicotine either in the form of a nicotine free base or a nicotine salt in either propylene glycol or vegetable glycerin or a combination of both with one or more flavorants added to taste. The resulting liquid may be sprayed, brushed, added in drops onto the pouch with a dropper or something that releases small liquid portions (e.g., drops), or otherwise applied to the pouch before the pouch is inserted in the user's mouth.

To summarize, the present disclosure is directed to an oral pouch, the active material for which is entirely and completely customizable by the end user of the pouch. The pouch as sold contains a carrier capable of sorbing active material but no active material.

Having described preferred embodiments of a new and improved oral pouch product it is believed that other modifications, variations, and changes will be suggested to those skilled in the art in view of the teachings set forth herein. It is therefore to be understood that all such variations, modifications, and changes are believed to fall within the scope of the invention as defined by the appended claims. Although specific terms are employed herein, they are used in a generic and descriptive sense only and not for purposes of limitation.

What is claimed is:

1. A method of delivering active materials to the mouth of a consumer by means of a consumer-customizable pouch comprising:
offering for sale an oral pouch of saliva-permeable pouch material containing a carrier capable of sorbing different active materials;
wherein the pouch as sold contains none of the active materials;
by the consumer:
adding active material of the consumer's choice to the pouch such that the added active material is sorbed by the carrier in the pouch; and
placing the pouch with the sorbed active material in the consumer's mouth.

2. The method of claim 1, wherein adding the active material(s) comprises delivering that active material as a liquid to and through the pouch material.

3. The method of claim 2 wherein the liquid active material is delivered as drops of liquid dripped or sprayed onto, soaked into, injected into, or poured on the pouch material.

4. The method of claim 1 wherein the carrier material is selected from the group consisting of microcrystalline cellulose, cornstarch, beet pulp fiber, silicon dioxide, calcium polycarbophil, and calcium carbonate and combinations thereof.

5. The method of claim 4 wherein active materials are medicaments, pharmaceuticals, stimulants. and essential oils.

6. The method of claim 5 wherein the pouch is manufactured from a non-woven material.

7. The method of claim 4 wherein the pouch product is sold in a container containing multiple identical pouches.

8. The method of claim 1 further comprising providing with the oral pouch as sold instructions directing consumers to add active materials of their choice to and through the oral pouch material.

9. An oral pouch product of saliva-permeable pouch material for delivering active materials to the mouths of consumers when contacted by saliva, wherein said oral pouch product as sold to consumers contains a carrier capable of sorbing the active materials but no active material, and wherein said pouch as sold is customizable to permit addition of active material thereto by consumers.

10. The oral pouch product of claim 9 further comprising instructions, accompanying the oral pouch product as sold, directing consumers to add active material of their choice to and through the oral ouch material.

11. The oral pouch product of claim 9 wherein the active materials are medicaments, pharmaceuticals, stimulants and essential oils.

12. The oral pouch product of claim 9 wherein the carrier material is selected from the group consisting of microcrystalline cellulose, cornstarch, beet pulp fiber, silicon dioxide, calcium polycarbophil, and calcium carbonate and combinations thereof.

13. A method of marketing an oral pouch product comprising selling an oral pouch product of saliva-permeable pouch material containing a carrier for active material capable of being by the carrier material, said pouch product as sold being characterized by containing no active material and being customizable by consumers adding active material of the consumers' choice to the oral pouch product.

14. The method of claim 13 further comprising instructing consumers to add the active material as liquid applied to and through the pouch material.

15. The method of claim 13 wherein the carrier material is selected from the group consisting of microcrystalline cellulose, cornstarch, beet pulp fiber, silicon dioxide, calcium polycarbophil, and calcium carbonate and combinations thereof.

16. The method of claim 15 wherein active material are medicaments, pharmaceuticals, stimulants and essential oils.

17. The method of claim 13 wherein the pouch product is sold in a container containing multiple identical pouches.

18. The method of claim 13 wherein the oral pouch material is non-woven material.
